(19) United States
(12) Patent Application Publication
(10) Pub. No.: US 2008/0058506 A1
(43) Pub. Date: Mar. 6, 2008

(54) Method and Device for Marking Biomolecules

(76) Inventors: Alexander Azzawi, Mainz (DE); Stefan Derschum, Wendelsheim (DE)

Correspondence Address: NORRIS, MCLAUGHLIN & MARCUS, PA, 875 THIRD AVENUE, 18TH FLOOR, NEW YORK, NY, 10022, US

(21) Appl. No.: 10/583,524

(22) PCT Filed: Oct. 28, 2004
(86) PCT No.: PCT/EP04/12172

§ 371 Date: Oct. 17, 2006

(30) Foreign Application Priority Data
Dec. 20, 2003 (DE) 103 50 484.2

(51) Int. Cl. C07K 14/00; B01F 15/04; C07H 5/06

(52) U.S. Cl. 530/408; 366/142; 530/402; 530/409; 530/410; 536/55.3

(57) ABSTRACT

The invention relates to a method which is used to efficiently mark proteins with the help of a micromixer, in addition to a device which enables said inventive method to be carried out in an economical manner and with additional auxiliary agents.

The invention relates to a process for efficiently labeling biomolecules, preferably proteins, nucleic acids and saccharides, with the aid of a micromixer, and also to an apparatus with which the process according to the invention can be carried out with a low demand for further assistants. The labeling reaction in the micromixer is superior to conventional methods for labeling biomolecules.

It is known that biomolecules, especially proteins, frequently have to be provided with label molecules for sensitive and specific detection. These label molecules may be dyes, electrochemically active compounds or else proteins themselves, such as peroxidase or green fluorescent protein (GFP). Such labelings are described, for example, by M. Brinkley in Bioconjugate Chemistry 1992, 3 2-13 and by R. P. Haugland in Methods in Molecular Biology 1995, 45, 205-221.

One means of labeling is the reaction of a biomolecule which, for the purposes of specific labeling, may also be present in modified form with an activated form of the label molecule. Typically, amino or sulfhydryl groups present on the biomolecule are labeled.

For the labeling of amino groups, for example the E-amino groups of the amino acid lysine present in a protein, a label molecule is provided with an appropriate activated functionality. This functionality may, for example, be an isothiocyanate (ITC) or a carboxylic acid function on the label molecule activated by an N-hydroxysuccinimide group (NHS) or a tetrafluorophenyl group (TFP). An NHS ester then reacts, for example, with a primary amino group to give the corresponding amide according to the following reaction:

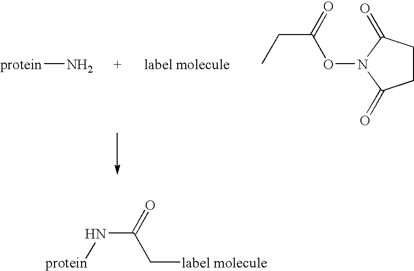

For the activation of sulfhydryl groups, appropriate maleimides are used.

For the labeling of biomolecules such as proteins, suitable activated derivatives of fluorescence labels are supplied in a large number by various manufacturers. When the methods specified for the labeling of proteins are compared, substantial agreement between the different methods is found: the protein is dissolved in an amine-free buffer (pH 7 to 9) in a concentration of about 10 mg/ml. To improve the solubility, 5% dimethyl sulfoxide (DMSO) may also be added. Between 2 and 10 molar equivalents of dyes, dissolved in DMSO, are then added and the reaction is stirred at room temperature for from 5 minutes to 2 hours. Subsequently, the reaction is stopped by adding a hydroxylamine solution or by the removal of the free dye, for example by gel permeation chromatography. However, in the labeling of biomolecules customary to date, especially of proteins, for example with dyes or fluorescence labels, there can be problems because an insoluble precipitate occasionally forms after the reaction with the biomolecule. This precipitate is attributable to overlabeling with the label. The overlabeled molecules cannot be employed for the further use and are thus lost.

In the case of irregular labeling of biomolecules, there can likewise be very high labeling of individual molecules. This leads to self-quenching of the fluorescence in these molecules and hence to a lower fluorescence intensity of the entire sample.

It is therefore an object of the invention to find a process in which, if possible, each biomolecule reacts with exactly the desired number of label molecules and to provide an apparatus suitable for this process. The protein or the biomolecule and the label molecule thus have to be mixed rapidly with one another in quantitative ratios determined beforehand in very small volume compartments. Subsequently, it has to be ensured that the molecules are in contact for a sufficiently long time to react with one another. After this time, the reaction is terminated to prevent side reactions.

A process has now been found for labeling biomolecules, preferably proteins, nucleic acids or saccharides, which bear free reactive groups, such as free amino, thiol, alcohol, aldehyde/ketone and/or carboxylic acid groups, with a label compound which reacts to form a covalent bond, in which solutions of both compounds, i.e. biomolecule solution and solution of the label compound, are fed in defined quantitative flow rates to a micromixer, preferably a static micromixer, and mixed intensively there. Subsequently, the reaction mixture is preferably fed into a delay structure, where it remains for a time predetermined by the volume of the delay structure and the flow rate of the reaction mixture. After a time predefined by the reaction conditions, the reaction is terminated.

The proteins, nucleic acids and/or saccharides used may be all common compounds, for example enzymes, membrane proteins, antibodies, deoxyribonucleic acid (DNA), RNA, polysaccharides.

According to the invention, the use of micromixers and delay structures considerably increases the efficiency of the labeling reaction over all methods known to date owing to the better mixing and metering of the reactants and the very precisely adjustable and narrow delay spectrum.

For the process according to the invention, it is possible to use all known static micromixers. Preference is given here to using static mixers which are flowed through continuously, for example multilamination mixers. These include, for example, stack mixers, slotted plate mixers, i.e. mixers in which streams of two different liquids are fanned out and the substreams are combined alternately by an interdigital configuration, or else comb mixers in which the two fluid streams to be mixed are fanned out into a multitude of thin lamellae or films, and these lamellae are subsequently intermixed alternately, so that diffusion and secondary flows lead to rapid mixing. These are obtainable, for example, from the product range of Ehrfeld Mikrotechnik BTS GmbH (EMB).

Likewise possible are V-type mixers from the Forschungszentrum Karlsruhe (FZK), split and recombine mixers, for example cascade mixers or faceted mixers (EMB) in which the solution streams are divided into smaller streams and these small streams are combined and divided repeatedly, caterpillar mixers from the Institut fur Mikrotechnik Mainz (IMM), or else mixers with cross-sectional narrowing such as focus mixers or cyclone mixers (IMM), or else jet mixers from Synthesechemie, and impingement jet mixers (IMM) or valve mixers (EMB).

For the process according to the invention, preference is given to using a micromixer in which the attachments and feed lines are configured such that only a small volume up to the inlet or outlet of the actual microstructure has to be flowed through (low-dead volume micromixer). This volume is preferably below 10% of the volume of the solutions to be mixed, more preferably below 1%, i.e., for example, below 5 μl for an amount of solution of 500 μl. A particularly suitable multilamination micromixer is one produced from PEEK (polyether ether ketone). In the examples, the capillary inlets of the feed lines are attached immediately opposite the orifices of the microstructure, so that this further minimized the dead volume.

In a preferred embodiment of the invention, micromixers with channel widths of less than 100 μm are used.

In such a micromixer, the biomolecule and the label molecule solutions are metered in by means of precise and low-pulsation pumps, for example syringe pumps. The reaction solution thus mixed is preferably subsequently fed into a delay structure with predefined delay time.

Alternatively, the solutions can also be metered into the micromixer via two syringes coupled to one another.

The mixing device preferably contains two orifices for attachment of metering syringes, the actual microstructured mixing structure, a short capillary-like structure with which a certain residence time and a pressure drop for easier metering is realized, an outlet for the mixed reaction solution, and recesses so that the mixing apparatus can be attached reliably to the customary reaction vessels, for example the reaction vessels (e.g. 0.5-2 ml) from Eppendorf or the screwcap tubes (e.g. 2-50 ml) from Greiner-Bio One.

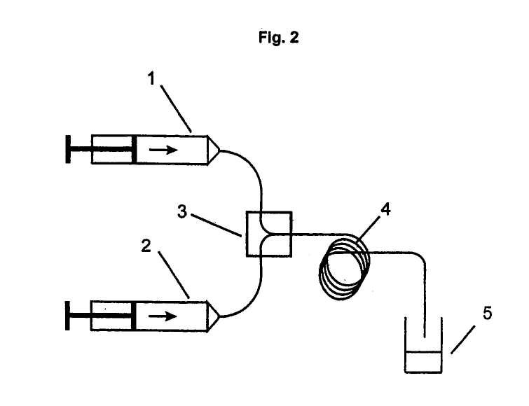

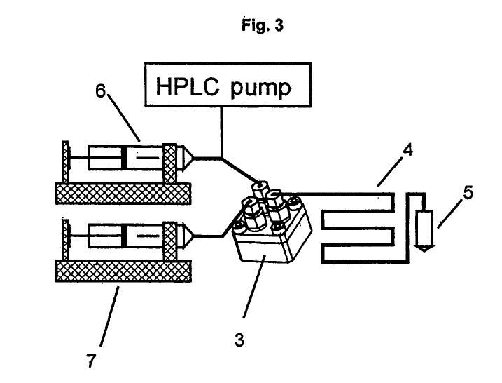

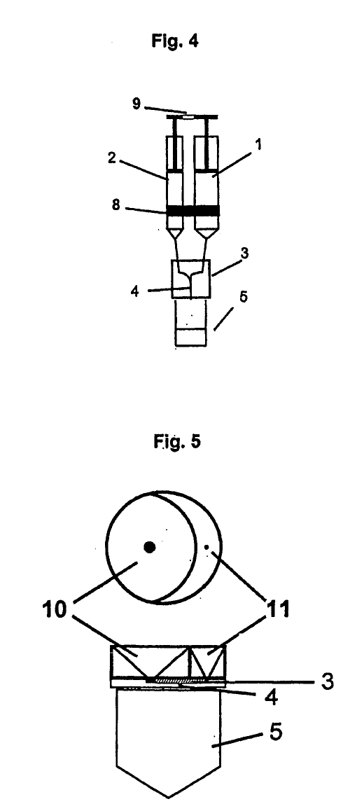

For the metering of the solutions into the mixing apparatus, it is possible to use two commercial syringes; see, for example, FIG. 2 or 3. The syringes are charged with the solutions. The volume of the syringes is guided preferably by the desired metering ratio of the two solutions. After they have been charged, the syringes are attached to the appropriate orifices of the mixing apparatus. In one embodiment of the invention, the two barrels and plungers of the syringe are connected to one another with small auxiliary devices, so that both plungers are pushed downward at the same speed when the connected plungers are pressed down. This ensures that a volume ratio, corresponding to the ratio of the diameters of the syringe cylinders, of the two reaction solutions is metered into the mixing apparatus; see FIG. 4.

The mixing apparatus may be an apparatus for multiple use. However, it is also possible by means of an inexpensive production process, for example by injection molding, to produce a large number of inexpensive mixing apparatuses which are intended for single use.

Alternatively to the processes already described, metering into the micromixer via centrifugal force is also possible, as illustrated by way of example in FIG. 5.

For this purpose, the mixing apparatus preferably contains two reservoirs on the upper side (10/11), the actual microstructured mixing structure (3), a short capillary-like structure with which a certain residence time and a pressure drop for easier metering can be realized, an outlet for the mixed reaction solution, and recesses so that the mixing apparatus can be attached reliably to the customary reaction vessels.

To meter the solutions into the mixing apparatus, the two solutions are introduced into the reservoirs (10/11). The size ratio of the reservoirs corresponds to the volume ratio of the solutions typically used in the reaction. The reservoirs are connected to the mixing structure (3) via small, precisely defined orifices. The dimension of the orifice and of the feed lines is preferably selected such that only when a force acts on the solutions in the reservoir are the two solutions metered into the mixing structure in the desired ratio.

The force can be generated by the centrifugation of the filled mixing apparatus attached to a collecting vessel, for example a 2 ml reaction vessel, in a laboratory centrifuge. Preference is given to the embodiment in which the two orifices of the reservoir are aligned relative to the mixing structure precisely at right angles to the axis of rotation of the centrifuge, so that the same force acts on both solutions. In order to enable easier alignment, the mixing apparatus is provided with appropriate markings.

Equally, the force can be generated by applying a pressure on the upper side of the reservoir or by applying a reduced pressure at the outlet of the mixing apparatus.

The mixing apparatus may likewise be an apparatus for multiple use. However, it is also possible by means of an inexpensive production process, for example by injection molding, to produce a large number of inexpensive mixing apparatuses which are intended for single use.

The inventive apparatus for performing a process for labeling biomolecules bearing reactive groups, preferably proteins, therefore preferably contains two reservoirs for liquids which optionally have a metering unit, preferably two syringes, a micromixer, optionally a delay structure and optionally any collecting device for the product.

In the context of the invention, delay structures are delimited volumes which can be flowed through within a predefined time owing to their internal volume, for example capillaries or else micromixers. It is possible for different delay structures to be used which each feature a very narrow delay time distribution and have low dead volumes. In the simplest case, the delay structure consists of a capillary of predefined length, but it is also possible to use other volumes or arrangements with uniform flow. It is likewise possible to use delay structures in which the mixture is pumped in circulation, in which case a micromixer is optionally inserted into the circuit. The latter is true in particular when two phases and phase separation are present.

After passing through the delay structure, the reaction solution is collected. The reaction can be stopped by metering in a further reagent by means of a further micromixer, by thermal treatment with a micro-heat exchanger or by adding the reaction mixture dropwise to a collecting vessel containing an appropriate termination reagent.

The label compound used is preferably a dye molecule which bears a reactive group, i.e. free amino, thiol, alcohol, aldehyde/ketone and/or carboxylic acid groups. These label compounds bearing reactive groups react to form covalent bonds, as described, for example, in M. Brinkley in Bioconjugate Chemistry 1992, 3, 2-13 and by R. P. Haugland in Methods in Molecular Biology 1995, 45, 205-221.

The labeling of alcohols in biomolecules, for example proteins such as serine, threonine and tyrosine, is done preferably by means of sulfonyl chlorides, for example dansyl chloride (5-dimethylaminonaphthalene-1-sulfonyl chloride) or the oxidation with sodium periodate to give the aldehyde and subsequent labeling with hydrazine derivatives of the dyes, for example hydrazides, semicarbazides, carbohydrazides, for example fluorescein-5-thiosemicarbazide.

Aldehydes/ketones are labeled preferably with amines (Schiff bases) or with hydrazine derivatives to give the hydrazide, while carboxylic acids are preferably labeled with hydrazine derivatives to give the hydrazide.

It is particularly preferred that the label molecule bears a reactive group which reacts with the biomolecules bearing free amino, thiol, alcohol and/or aldehyde/ketone to form covalent bonds, and the label molecule additionally catalyzes a chemical reaction which leads to an easily detectable color change or change in the redox potential of its substrate, for example peroxidases and alkaline phosphatases.

Particularly preferred reactive groups are amino and/or thiol groups.

After leaving the delay structure, the labeling reaction can be terminated by adding a compound which reacts rapidly with the label molecules yet to be converted. These are commercial compounds containing amino and/or thiol groups, for example hydroxylamine, glutathione or mercaptoethanol.

After leaving the delay structure, the reaction can likewise be terminated by chromatographic removal of the label molecules yet to be converted. Suitable for this purpose are all commercial chromatography processes, for example gel permeation chromatography with, for example, Sephade® G or Biogel® P, or ion exchange chromatography, affinity chromatography, reversed phase chromatography (HPLC) and solid-phase extraction.

In a further embodiment of the invention, the reaction, after leaving the delay structure, can be terminated by a thermal treatment of the reaction solution. This is done preferably by cooling the reaction solution, preferably by means of a microstructured heat exchanger, or by heating the reaction solution, preferably by means of a microstructured heat exchanger.

The process according to the invention leads to higher yields and to improved sample quality, since rapid and thorough mixing of the two reactants takes place in the micromixer and the delay time in the delay structure can be adjusted very precisely.

The invention is illustrated in detail by the figures appended and the examples which follow without being restricted thereto.

The figures show:

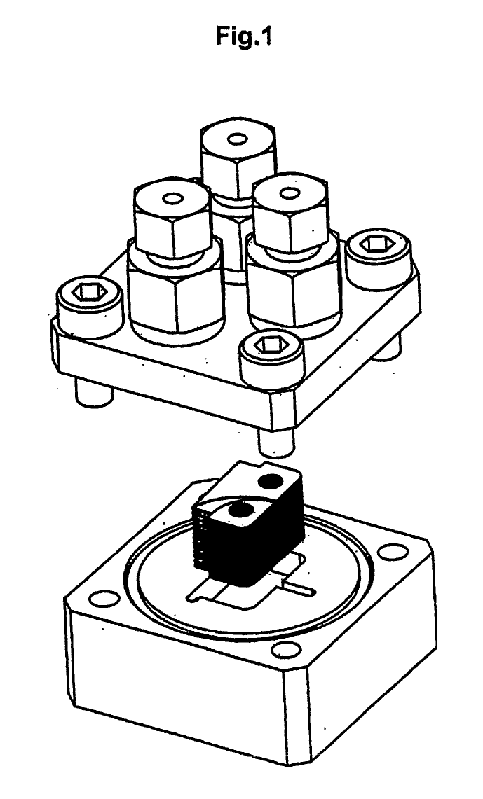

FIG. 1 the exploded diagram of a low-dead volume micromixer,

FIG. 2 the schematic experimental construction with the micromixer,

FIG. 3 the complete experimental apparatus,

FIG. 4 the schematic experimental construction with a micromixer which enables simultaneous metering via 2 syringes and

FIG. 5 the schematic experimental construction with metered addition from 2 reservoirs via centrifugal force or pressure.

### EXAMPLES

### Example 1

The intention was to label anti-human IgG antibodies (from the goat) with fluorescein isothiocyanate (FITC). The antibodies (supplier: Sigma, Saint Louis) were dissolved in 0.1 M sodium hydrogencarbonate buffer (pH 8.5) (10 mg/ml). 10 mg of FITC (supplier: Molecular Probes, Eugene) were dissolved in 1 ml of DMSO. In addition, a 1.5 M hydroxylamine solution (pH 8.4) was prepared to stop the reaction.

The conventional labeling method, as specified in the description supplied with the fluorescent label, is compared with the inventive labeling in the micromixer.

### A1 and A2—Conventional Labeling Process

Two batches of 75 μl of antibody solution in each case were introduced into two 1.5 ml reaction vessels. 37.5 μl of the dye solution were added to one batch (A1), 10 μl to the other (A2). The vessels were shaken vigorously at room temperature for 1 h. Subsequently, 100 μl of the hydroxylamine solution in each case were added and the vessels were again shaken at room temperature for 20 min. Thereafter, the free dye was removed by means of gel permeation chromatography (PD-10 columns, supplier: Amersham, eluent: phosphate-buffered saline (PBS)).

The antibody solutions thus purified were analyzed by UV/V is spectroscopy.

B—Inventive Labeling Process

The two syringes (1, 2) shown in FIG. 2 were connected via capillaries to a micromixer (3). In addition, a flush line shown in FIG. 3 was attached to the capillary of one of the syringes and was initially closed with a clamp. This flush line was attached to an HPLC pump which was supplied with water (bidistilled). The micromixer (3) was in turn connected to a capillary (4) with a length of 28 cm. At the end of the capillary, a collecting vessel (5) was provided, in which the reaction solution was collected.

The inventive labeling process was then carried out in the following manner:

The antibody solution and the dye solution were each drawn up in a 1 ml syringe and clamped into one of the syringe pumps (6) and (7) in each case.

Batch B1:

75 μl of protein solution (flow rate: 150 μl/min) and 37.5 μl of dye solution (flow rate: 75 μl/min) were metered into the mixer within 30 seconds. Subsequently, the clamp of the flush line was opened and the solution still present in the capillary was expelled by means of an HPLC pump with a flow rate of 0.2 μl/min. The collecting vessel (5) was closed and shaken at room temperature for one hour.

Batch B2:

75 μl of protein solution (flow rate: 150 μl/min) and 10 μl of dye solution (flow rate: 20 μl/min) were metered into the mixer within 30 seconds. The batch was treated further as described in B1.

The two batches of the B series were, after shaking for one hour, admixed with 100 μl of a hydroxylamine solution in each case and shaken again at room temperature for 20 min. Subsequently, the free dye was removed by means of gel permeation chromatography (PD-10 columns, supplier: Amersham, eluent PBS).

The antibody solutions thus purified were analyzed by UV/V is spectroscopy.

In all four batches, between 0.6 and 0.7 mg of labeled antibody were obtained from the 0.75 mg of antibodies used after the purification of the reactions. The degrees of labeling (molecules of dye per molecule of protein) are 8.62 for A1, 6.32 for A2, 4.91 for B1 and 2.30 for B2.

The labeled antibodies were tested for their suitability for staining granulocytes.

It was found that the samples of the A series had a very high background. The samples of the B series exhibited a low background. The cell nucleus and the mitosis spindles were, as expected, very readily visible.

The use of the micromixer thus allowed a distinctly improved sample quality to be obtained. This is attributable to more uniform labeling of the antibodies.

### Example 2

In the reaction shown in this example, a protein is provided with a fluorescent label. The NHS ester of the fluorescent label was used in order to react unspecifically with the ε-amino groups of the amino acid lysine present in the protein.

0.27 μM of protein were dissolved in 1800 μl of NaHCO₃ buffer (0.1 M, pH 8). For better dissolution, 100 μl of DMSO were added. 1.08 μM of dye NHS ester were dissolved in 140 μl of DMSO. In the micromixer, the protein and the dye solutions were metered in by means of syringe pumps via ⅙″ capillaries. The total flow rate was 400 μl/min.

At the outlet of the mixer was disposed a capillary which was sufficiently long that the delay time of the reaction solutions in the system was 5 minutes. Subsequently, the solution was added dropwise to a collecting vessel (5) which was filled with the stop solution (hydroxylamine solution). Once the appropriate amounts of protein and dye solution had been metered in, the volume metered in last had to be forced through the delay capillary for another 5 min. This was done with the aid of an HPLC pump which pumped water through the capillary with a flow rate of 400 μl/min.

After the end of the reaction, the label batch collected in the stop solution was stirred for another 15 minutes and subsequently centrifuged in a bench centrifuge (Eppendorf 5804 R) at room temperature and 13 200 rpm in order to remove insoluble constituents. However, barely any insoluble constituents were observed in this reaction.

The mixture was applied to PD-10 columns (supplier: Amersham) with a pipette. The eluate was collected in freeze-drying vials, frozen and subsequently lyophilized.

In relation to the amount of protein used, a yield of 86.6% with a degree of labeling of 2.9 was achieved. The standard method recommended by the manufacturers achieved only 45% yield with a degree of labeling of 2.7.

In addition, the sample prepared in the micromixer exhibited slightly improved activity in the subsequent activity test compared to the conventionally prepared sample.

### REFERENCE NUMERAL LIST

1 Syringe 1

2 Syringe 2

3 Micromixer

4 Capillary line

5 Collecting vessel

6 Syringe pump 1

7 Syringe pump 2

8 Connections of the two barrels

9 Connections of the two plungers

10 Reservoir 1

11 Reservoir 2

1. A process for labeling biomolecules bearing free reactive groups with a label compound which reacts to form a covalent bond, comprising feeding solutions of both compounds are fed in defined quantitative flow rates to a micromixer, and mixing intensively there, then optionally feeding the reaction mixture into a delay structure, and retaining there for a time predetermined by the volume of the delay structure and the flow rate of the reaction mixture, and terminating the reaction after a time predefined by the reaction conditions.

2. The process as claimed in claim 1, wherein the free reactive groups are amino, thiol, alcohol, aldehyde/ketone and/or carboxylic acid groups.

3. The process according to claim 1, wherein the biomolecules are proteins, nucleic acids and/or saccharides.

4. The process according to claim 1, wherein the micromixer is a micromixer with channel widths of less than 100 μm is used.

5. The process according to claim 1, wherein the micromixer is a multilamination mixer or a split and recombine mixer.

6. The process according to claims 1, wherein the delay structure is a capillary of predefined volume or another volume with uniform flow or an arrangement with uniform flow.

7. The process according to claim 1, wherein the reaction mixture is pumped in circulation in the delay structure used, a micromixer optionally being inserted into the circuit.

8. An apparatus for performing the process according to claims 1.
